(19) United States
(12) Patent Application Publication
(10) Pub. No.: US 2023/0000754 A1
(43) Pub. Date: Jan. 5, 2023

(54) Liquid Foundation Using Starch as Filler and Preparation Method thereof

(71) Applicant: Jiangnan University, Wuxi (CN)

(72) Inventors: Yan Hong, Wuxi (CN); Wenwen Zhu, Wuxi (CN); Zhengbiao Gu, Wuxi (CN); Li Cheng, Wuxi (CN); Zhaofeng Li, Wuxi (CN); Caiming Li, Wuxi (CN); Xiaofeng Ban, Wuxi (CN)

(21) Appl. No.: 17/903,273

(22) Filed: Sep. 6, 2022

Related U.S. Application Data
(63) Continuation of application No. PCT/CN2021/096581, filed on May 28, 2021.

(30) Foreign Application Priority Data
Dec. 8, 2020 (CN) 2020114611858

(51) Int. Cl. A61K 8/73; A61K 8/02; A61K 8/19; A61Q 1/12; A61Q 1/02

(52) U.S. Cl. CPC A61K 8/732; A61K 8/0241; A61K 8/19; A61Q 1/12; A61Q 1/02; A61K 2800/43

(57) ABSTRACT

The disclosure discloses a liquid foundation using starch as a filler and a preparation method thereof, belonging to the technical fields of deep processing of starch and preparation of cosmetics. In the disclosure, starch is modified by an OSA reagent and then homogenized and micronized to obtain a starch filler; powder materials and the water phase in the formula of the liquid foundation are sequentially added to the oil phase at a certain temperature; the mixture is sheared at low speed, homogenized, and cooled to room temperature; and the starch filler is added to prepare the liquid foundation using starch as a filler, wherein the formula includes 10-20 parts of oil phase, 65-75 parts of water phase, 5-10 parts of toner, and 5-15 parts of starch filler. The starch filler in the disclosure is hypoallergenic, environmental and safe, and the liquid foundation containing the starch filler is refreshing and not easy to clog pores, solves the problems of traditional fillers which are thick and greasy, unbreathable, easy to aggravate facial acne, and the like, and is in line with the current propensity to consume of consumers for green and safe beauty products.

### TECHNICAL FIELD

The disclosure relates to a liquid foundation using starch as a filler and a preparation method thereof, and in particular relates to a preparation method of an emulsified liquid foundation and the characteristics of the starch filler, belonging to the technical fields of deep processing of starch and preparation of cosmetics.

### BACKGROUND

With people's gradually growing awareness of beauty and skin care, the requirements for beauty products are also getting higher and higher. Especially for a foundation product used in the “first step of makeup”—a liquid foundation, its quality and safety have become the primary considerations for consumers' choice. At present, the liquid foundations on the market mostly use talcum powder, silicon dioxide, polymethyl methacrylate, nylon powder, silicon elastomer, etc. as fillers, most of which are synthetic ingredients or mineral ingredients, are thick and greasy when applied, will cause the facial skin to become not breathable and greasier after application, and will clog the pores and cause problems such as spots and acne on the skin.

As a natural and renewable environmental resource, starch is hypoallergenic and biodegradable, and is very suitable as a powder raw material in cosmetics. Also, starch is easily modified, and starch fillers suitable for emulsified liquid foundation products can be controllably obtained by different methods and degrees of modification.

In the prior art, starch or modified starch is selected as a filler in some cases, but still ingredients such as kaolin need to be added and used as a filler simultaneously to ensure the stability and applicability of a liquid foundation, so there may still be problems such as heavy texture, greasy feel, and light smooth feeling.

Therefore, it is of great significance to research and develop non-irritating green and natural modified starch to completely replace talcum powder, silicon elastomer and other fillers in cosmetic foundations. Moreover, it is of great significance to develop the application of modified starch in the field of fine chemicals.

### SUMMARY

To solve the problems existing in the prior art, the disclosure provides a liquid foundation using starch as a filler and a preparation method thereof. In the disclosure, modified starch is obtained by two modification methods of esterification and micronization, so that the modified starch can be stably dispersed in a liquid foundation system, has good stability and applicability without the aid of other fillers, reduces costs and performs well.

The first objective of the disclosure is to provide a liquid foundation using starch as a filler, including the following ingredients in parts by mass:

| | |
|---|---|
| Oil phase | 10-20 parts |
| Water phase | 65-75 parts |
| Toner | 5-10 parts |
| Starch filler | 5-15 parts |

where the OSA degree of substitution of the starch filler is 0.02-0.05, and the average particle size is 5-12 μm.

Further, in the liquid foundation using starch as a filler, the composition of water phase raw materials includes the following ingredients in parts by mass: 0.1-0.5 part of p-hydroxyacetophenone, 1-3 parts of triethanolamine, 2-4 parts of propylene glycol, 0.1-0.5 part of sodium carboxymethyl cellulose and 57-71.8 parts of water.

Further, the water is preferably deionized water or high-purity water.

Further, in the liquid foundation using starch as a filler, the composition of the oil phase includes the following ingredients in parts by mass: 1-3 parts of stearic acid, 1-3 parts of glycerol monostearate, 0.5-2 parts of lanolin, and 2-17.5 parts of white mineral oil.

Further, in the liquid foundation using starch as a filler, the composition of the toner includes the following ingredients in parts by mass: 0.5-2 parts of an inorganic colorant and 3-9.5 parts of titanium dioxide.

Further, the inorganic colorant includes one or more of iron oxide yellow, iron oxide red, and iron oxide black.

Further, when the inorganic colorant includes iron oxide yellow and iron oxide red, preferably, the mass ratio of the iron oxide yellow to the iron oxide red is 4:1.

Further, the apparent viscosity of the liquid foundation is 1000-3000 mPa·s.

Further, under the action of high-speed shearing, the liquid foundation exhibits the characteristic of shear thinning, but the rheological index n is less than 0.5, the shear resistance is high, and the consistency coefficient K is 10-40 Pa·s.

The second objective of the disclosure is to provide a preparation method of the liquid foundation using starch as a filler, and the method includes the following steps:

1) Weighing raw materials according to the formula, and heating the water phase and oil phase raw materials to 75-80° C. to completely dissolve the raw materials;

2) Under a stirring state at 70-75° C., adding a toner powder mixed evenly to the oil phase;

3) Then adding the water phase raw materials under high-speed stirring, mixing the raw materials evenly for 10-20 min, and cooling the mixture to 20-30° C.; and

4) Adding a filler at 20-30° C., stirring the mixture for 5-10 min, and then performing discharging and packing after stirring.

Further, the stirring speed in step (2) is 2000-5000 rpm.

Further, the speed of the high-speed stirring in step (3) is 8000-12000 rpm.

Further, the speed at which the water phase raw materials are added in step (3) is preferably slow.

Further, the stirring speed in step (4) is 2000-5000 rpm.

The third objective of the disclosure is to provide a starch filler, the OSA degree of substitution of the starch filler is 0.02^{˜}0.05, and the average particle size is 5-12 μm.

Further, the starch filler is obtained by modifying starch with an OSA reagent (aluminum octenyl succinate) and then performing homogenization and micronization.

Further, the method specifically includes: under the reaction conditions of pH 8.0-8.8 and 30-40° C., adding an OSA reagent which is 5-9% of the mass of starch to starch milk to react for 3-5 h; and after neutralizing and washing, micronizing the mixture using a high-pressure homogenizer, homogenizing the mixture 20-40 times under a pressure of 40-70 MPa, and centrifugally drying, pulverizing and sieving the mixture.

Further, the starch milk is obtained by mixing starch with water, where the starch is any one or more of corn starch, wheat starch, potato starch, tapioca starch and corresponding waxy starch thereof.

Further, the concentration of the starch milk is 30-40% (w/w, d.b).

The fourth objective of the disclosure is a daily chemical product containing the starch filler.

The fifth objective of the disclosure is an application of the starch filler in the field of fine chemicals.

Compared with the prior art, the disclosure has the following beneficial effects:

1. The disclosure provides a formula and a preparation method of a new-formula liquid foundation using starch as a filler. In the disclosure, compound modified starch is used as a filler, and starch is modified by esterification and micronization, so that the prepared starch has better hydrophobicity and emulsifying property, and can be used as a filler in a liquid foundation. The starch filler of the disclosure can be stably dispersed in the liquid foundation system, and improve the storage stability and persistence of the liquid foundation, and the liquid foundation is less prone to water-oil separation. In addition, the disclosure uses the green and non-allergenic starch as the filler of the liquid foundation. As a new green filler, different from the traditional fillers with a heavy feeling, starch has the advantages of easy applicability and good adhesion.

2. The starch filler for liquid foundations prepared by the disclosure has a narrow particle size distribution range, good hydrophobicity and good emulsifying stability, plays the roles of filling, soft focus and thickening in liquid foundations, is a green filler with green, safe and non-allergenic properties, can completely replace fillers such as talcum powder and silicone elastomer in traditional liquid foundations, improves the emulsifying stability of liquid foundations, and can also absorb oil secreted by the skin to make the makeup more persistent and not easy to take off.

### BRIEF DESCRIPTION OF FIGURES

FIG. 1 is the static rheological diagram of powder foundation samples of Examples 1-4 with a starch filler added.

FIG. 2 shows the dispersibility of the starch filler samples in the oil phases of Example 1 and Comparative Example 1.

FIG. 3 shows a photograph of the appearances and the oil separation rates of the liquid foundation samples (standing for 5 months) of Example 1 and Comparative Example 2.

### DETAILED DESCRIPTION

Implementations of the disclosure will be described in detail below with reference to the examples. However, those skilled in the art will understand that the following examples are merely illustrative of the disclosure and should not be construed as limiting the scope of the disclosure.

The examples in which specific conditions are not specified are carried out according to conventional conditions or conditions recommended by the manufacturer. Any reagents or instruments that are not indicated with the manufacturers are commercially available products.

Determination of degree of substitution of starch: Refer to GB 28303-2012 “Food Additive Starch Sodium Octenyl Succinate” for a determination method.

Determination of particle size of starch: An appropriate amount of starch sample is taken and placed in a tank of a laser particle size analyzer. After the sample is absorbed, sample injection is completed by the laser particle size analyzer, and a particle size distribution diagram and average particle size data are obtained by processing using software.

Determination of gelatinization characteristic of starch: 6% (w/w, d.b) starch milk is prepared, and measured by an RVA viscometer using a Standard 2 procedure specified by the American association of cereal chemistry (AACC).

Determination of contact angle of starch: 0.50 g of starch sample is taken and pressed into a tablet by a tablet press machine under 25 MPa, and then a three-phase contact angle of the sample surface to water in an oil phase is measured by a contact angle meter.

Determination of apparent viscosity of liquid foundation sample: A liquid foundation sample is added to a measuring cup, a rotor is installed to connect to a BROOKFIELD rotating viscometer, and the viscosity value is recorded at the 10th s, where the rotational speed is 60 r/min, and the rotor model is No. 29.

Determination of rheological properties of liquid foundation sample: An appropriate amount of liquid foundation sample is taken and placed on a bottom plate of an HR-3 rheometer, where the diameter of the plate is 4.0 cm, the setting gap is 1.0 mm, and the measurement temperature is 25° C. A static shear rheometry program is run: the shear rate (y) is set to increase progressively from 1 s⁻¹ to 100 s⁻¹, and the shear stress (t) of a sample is measured as a function of the shear v rate.

Determination of dispersion rate: 4 g of dried starch sample is weighed, 10 g of oil phase is added and mixed with the sample evenly, and the mixture is transferred into a mixing cylinder with stopper. The mixture is allowed to stand at room temperature, and the settling volume is read once every 1 h to calculate the dispersion rate.

Determination of oil separation rate of liquid foundation: 100 mL of liquid foundation sample is measured in a mixing cylinder with stopper. The liquid foundation sample is allowed stand at room temperature, the volume of the separated oil phase is read at regular intervals, and the oil separation of different samples is compared.

Sensory evaluation method of liquid foundation: The 5 makeup effects of a liquid foundation product, including applicability, skin adhesion, concealing effect, transparency, and skin tone brightening effect, are evaluated by self-assessment of consumers, and scores are obtained by quantitative descriptive analysis from a sensory evaluation panel (including 50 volunteers). The sensory indices of the liquid foundation are described in Table 1.

TABLE 1
Sensory evaluation indices and definitions

| Sensory | | | |
|---|---|---|---|
| attributes | Definition | Dimension range | Score |
| Applicability | The ease with which the product is applied | 15 (Easy) − 1 (Difficult) | 15, |
| | to the skin surface to an even state | | |
| Skin adhesion | Adhesion effect of the product on the skin | 15 (Good) − 1 (Poor) | 13, |
| | after application | | |
| Concealing | The ability to conceal the | 15 (Very well | 11, 9, |
| effect | original complexion of the skin after application of | concealment) − 1 (No concealment) | |
| | the product | | |
| Transparency | Whether the sense of | 15 (No sense | 7, 5, |
| | makeup is obvious on | of makeup) − 1 | |
| | the surface after application | (Obvious sense | |
| | of the product | of makeup) | |
| Brightness | Degree of obvious increase in skin surface | 15 (Increase) − 1 (Decrease) | 3, 1, |
| | brightness after application of the product | | |

In conjunction with examples, the liquid foundation using starch as a filler and preparation thereof of the disclosure will be further described below:

### Example 1

1. A liquid foundation using starch as a filler, including the following composition ingredients (where the inorganic colorant includes iron oxide yellow and iron oxide red in a mass ratio of 4:1):

| | |
|---|---|
| Stearic acid | 1 part |
| White mineral oil | 10 parts |
| Glycerol monostearate | 2 parts |
| Lanolin | 1 part |
| P-hydroxyacetophenone | 0.1 part |
| Triethanolamine | 1 part |
| Propylene glycol | 3 parts |
| Sodium carboxymethyl cellulose | 0.1 part |
| Deionized water | 66.8 parts |
| Titanium dioxide | 7 parts |
| Inorganic colorant | 1 part |
| Starch filler | 7 parts |

2. A preparation method of example 1 includes the following steps:

1) The raw materials were weighed according to the formula, and the water phase and oil phase raw materials were heated to 70° C. to completely dissolve the raw materials;

2) The oil phase was insulated at 75° C., and a toner powder mixed evenly was added under stirring;

3) The water phase raw materials were added under high-speed stirring and mixed evenly for 10 min, and the mixture was cooled to room temperature; and

4) The starch filler was added at 20° C., and the mixture was stirred for 10 min, and discharged and packed after stirring.

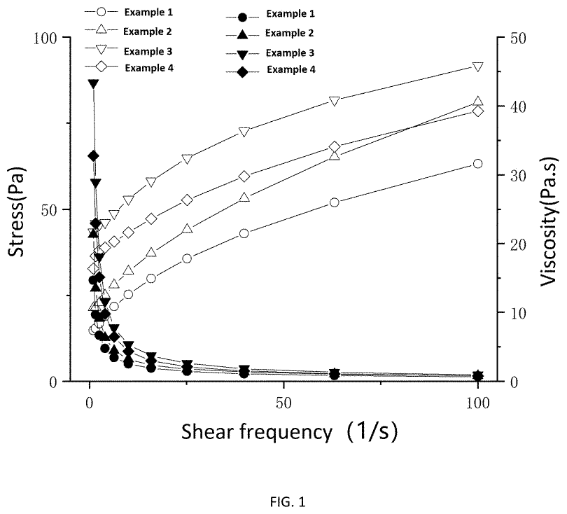

3. The relevant properties of Example 1 were measured. As shown by data in Table 2 and FIG. 1, the apparent viscosity is 1255 mPa·s; also, shown by static rheological fitting data, the liquid foundation obtained in Example 1 is shear-thinned, has a fluid index of 0.3282 (less than 0.5), and has a certain shear resistance; and the liquid foundation has a relatively small consistency coefficient K, which is 12.724 Pa·s, indicating that the liquid foundation prepared by the disclosure has certain fluidity and is easy to apply.

TABLE 2
Properties of emulsified liquid foundation sample

| | | Rheological properties | | |
|---|---|---|---|---|
| | Apparent | Consistency | | Coefficient of |
| Sample | viscosity/ mPa · s | coefficient K/(Pa · s) | Rheological index n | determination R² |
| Example 1 | 1255 | 12.724 | 0.3282 | 0.9801 |

4. A preparation method of the starch filler in Example 1 includes: under the reaction conditions of pH 8.5 and 35° C., 5 wt % of an OSA reagent was added to 35% (w/w, d.b) starch milk to react for 4 h; and the mixture was neutralized, washed, micronized using a high-pressure homogenizer, homogenized 20 times under a pressure of 40 MPa, centrifugally dried, pulverized and sieved to obtain compound modified starch for the filler. The filler in Example 1, that is, the compound modified starch, was characterized for the degree of modification with the results shown in Table 3, and measured for the relevant properties with the results shown in Table 4. It can be seen that the degree of substitution of OSA is 0.0263 and the average particle size is 10.24 μm.

TABLE 3
Degree of modification of starch for filler

| Characterization item | Example 1 | Example 2 | Example 3 | Example 4 |
|---|---|---|---|---|
| Degree of | 0.0263 | 0.0352 | 0.0421 | 0.03815 |
| substitution of OSA | | | | |
| Average particle | 10.24 | 6.82 | 10.58 | 7.94 |
| size/μm | | | | |

TABLE 4
Relevant properties of compound modified starch for filler

| | Gelatinization characteristic | | | |
|---|---|---|---|---|
| | | Peak | Final | |
| | Gelatinization | viscosity/ | viscosity/ | Contact |
| Sample | temperature/° C. | (mPa · s) | (mPa · s) | angle/° |
| Example 1 | 63.4 | 3880 | 1649 | 57.80 |

5. To further illustrate the beneficial effects of the disclosure, the liquid foundation sample was subjected to sensory evaluation, and the evaluation scores were shown in Table 5, where the control was a commercial liquid foundation product with talcum powder and bentonite as the fillers. It can be seen that the liquid foundation using starch as a filler prepared by the disclosure is close to the control sample in performance, and can basically reach the standard of the commercial product. The higher scores of Example 1 in terms of transparency and brightness indicate that the sample of Example 1 has better makeup effect.

TABLE 5
Sensory evaluation scores of emulsified liquid
foundation samples

| | | Skin | Concealing | Trans- | |
|---|---|---|---|---|---|
| | Applicability | adhesion | effect | parency | Brightness |
| Example 1 | 10.5 | 9.4 | 8.9 | 9.3 | 11.2 |
| Control | 10.8 | 10.0 | 9.5 | 8.9 | 9.6 |

### Example 2

1. A liquid foundation using starch as a filler, including the following composition ingredients (where the inorganic colorant includes iron oxide yellow and iron oxide red in a mass ratio of 4:1):

| | |
|---|---|
| Stearic acid | 2 parts |
| White mineral oil | 8 parts |
| Glycerol monostearate | 3 parts |
| Lanolin | 2 parts |
| P-hydroxyacetophenone | 0.2 part |
| Triethanolamine | 1 part |
| Propylene glycol | 2 parts |
| Sodium carboxymethyl cellulose | 0.1 part |
| Deionized water | 67 parts |
| Titanium dioxide | 6 parts |
| Inorganic colorant | 0.7 part |
| Starch filler | 8 parts |

2. A preparation method of example 2 includes the following steps:

1) The raw materials were weighed according to the formula, and the water phase and oil phase raw materials were heated to 75° C. to completely dissolve the raw materials;

2) The oil phase was insulated at 70° C., and a toner powder mixed evenly was added under stirring;

3) The water phase raw materials were added under high-speed stirring and mixed evenly for 15 min, and the mixture was cooled to room temperature; and

4) The starch filler was added at 25° C., and the mixture was stirred for 10 min, and discharged and packed after stirring.

3. The relevant properties of Example 2 were measured. As shown by data in Table 6 and FIG. 1, the apparent viscosity is 1820 mPa·s; also, shown by static rheological fitting data, the liquid foundation obtained in Example 2 is shear-thinned, has a fluid index of 0.2982, and has certain shear resistance; and the liquid foundation has a relatively small consistency coefficient K, has certain fluidity and is easy to apply.

TABLE 6
Properties of emulsified liquid foundation sample

| | | Rheological properties | | |
|---|---|---|---|---|
| | Apparent | Consistency | | Coefficient of |
| | viscosity/ | coefficient K/ | Rheological | determination |
| Sample | mPa · s | (Pa · s) | index n | R² |
| Example 2 | 1820 | 17.778 | 0.2982 | 0.9560 |

4. A preparation method of the starch filler in Example 2 includes: under the reaction conditions of pH 8.6 and 35° C., 7 wt % of an OSA reagent was added to 35% (w/w, d.b) starch milk to react for 4 h; and the mixture was neutralized, washed, micronized using a high-pressure homogenizer, homogenized 30 times under a pressure of 50 MPa, centrifugally dried, pulverized and sieved to obtain compound modified starch for the filler. The filler in Example 2, that is, the compound modified starch, was characterized for the degree of modification with the results shown in Table 3, and measured for the relevant properties with the results shown in Table 7. It can be seen that the degree of substitution of OSA is 0.0352 and the average particle size is 6.82 μm.

TABLE 7
Relevant properties of compound modified starch for filler

| | Gelatinization characteristic | | | |
|---|---|---|---|---|
| | Gelatinization | | | |
| | temperature/ | Peak viscosity/ | Final viscosity/ | Contact |
| Sample | ° C. | (mPa · s) | (mPa · s) | angle/° |
| Example 2 | 56.3 | 4258 | 1577 | 61.30 |

5. To further illustrate the beneficial effects of the disclosure, the liquid foundation sample was subjected to sensory evaluation, and the evaluation scores were shown in Table 8. It can be seen that the liquid foundation using starch as a filler prepared by the disclosure is close to the control sample in performance, and can basically reach the standard of the commercial product. Also, since the particle size of the starch added in Example 2 is smaller, the liquid foundation of Example 2 has better applicability and skin adhesion.

TABLE 8
Sensory evaluation scores of emulsified liquid foundation samples

| | | Skin | Concealing | Trans- | Bright- |
|---|---|---|---|---|---|
| | Applicability | adhesion | effect | parency | ness |
| Example | 11.4 | 11.5 | 8.9 | 10.7 | 12.1 |
| 2 | | | | | |
| Control | 10.8 | 10.0 | 9.5 | 8.9 | 9.6 |

### Example 3

1. A liquid foundation using starch as a filler, including the following composition ingredients (where the inorganic colorant includes iron oxide yellow and iron oxide red in a mass ratio of 4:1):
Stearic acid 1.5 parts

| | |
|---|---|
| White mineral oil | 9 parts |
| Glycerol monostearate | 2.5 parts |
| Lanolin | 1.5 parts |
| P-hydroxyacetophenone | 0.3 part |
| Triethanolamine | 1.5 parts |
| Propylene glycol | 3 parts |
| Sodium carboxymethyl cellulose | 0.1 part |
| Deionized water | 66.1 parts |
| Titanium dioxide | 8 parts |
| Inorganic colorant | 0.5 part |
| Starch filler | 10 parts |

2. A preparation method of example 3 includes the following steps:

1) The raw materials were weighed according to the formula, and the water phase and oil phase raw materials were heated to 80° C. to completely dissolve the raw materials;

2) The oil phase was insulated at 75° C., and a toner powder mixed evenly was added under stirring;

3) The water phase raw materials were added under high-speed stirring and mixed evenly for 20 min, and the mixture was cooled to room temperature; and

4) The starch filler was added at 25° C., and the mixture was stirred for 15 min, and discharged and packed after stirring.

3. The relevant properties of Example 3 were measured. As shown by data in Table 9 and FIG. 1, the apparent viscosity is 2150 mPa·s, and shown by static rheological fitting data, the liquid foundation obtained in Example 3 is shear-thinned, has a fluid index of 0.1644 and a relatively large consistency coefficient K within the limit, and has slightly poor fluidity, shear resistance and strong adhesion.

TABLE 9
Properties of emulsified liquid foundation sample

| | | Rheological properties | | |
|---|---|---|---|---|
| | Apparent | Consistency | | Coefficient of |
| | viscosity/ | coefficient | Rheological | determination |
| Sample | mPa · s | K/(Pa · s) | index n | R² |
| Example 3 | 2150 | 39.281 | 0.1644 | 0.9266 |

4. A preparation method of the starch filler in Example 3 includes: under the reaction conditions of pH 8.7 and 35° C., 9 wt % of an OSA reagent was added to 35% (w/w, d.b) starch milk to react for 4 h; and the mixture was neutralized, washed, micronized using a high-pressure homogenizer, homogenized 20 times under a pressure of 60 MPa, centrifugally dried, pulverized and sieved to obtain compound modified starch for the filler. The filler in Example 3, that is, the compound modified starch, was characterized for the degree of modification with the results shown in Table 3, and measured for the relevant properties with the results shown in Table 10. It can be seen that the degree of substitution of OSA is 0.0421 and the average particle size is 10.58 μm.

TABLE 10
Relevant properties of compound modified starch for filler

| | Gelatinization characteristic | | | |
|---|---|---|---|---|
| | Gelatinization | | | |
| | temperature/ | Peak viscosity/ | Final viscosity/ | Contact |
| Sample | ° C. | (mPa · s) | (mPa · s) | angle/° |
| Example 3 | 59.9 | 4190 | 1574 | 84.00 |

5. To further illustrate the beneficial effects of the disclosure, the liquid foundation sample was subjected to sensory evaluation, and the evaluation scores were shown in Table 11. It can be seen that the liquid foundation using starch as a filler prepared by the disclosure is close to the control sample in performance, and can basically reach the standard of the commercial product.

TABLE 11
Sensory evaluation scores of emulsified liquid foundation samples

| | | Skin | Concealing | Trans- | Bright- |
|---|---|---|---|---|---|
| | Applicability | adhesion | effect | parency | ness |
| Example 3 | 9.1 | 10.6 | 11.7 | 8.4 | 9.3 |
| Control | 10.8 | 10.0 | 9.5 | 8.9 | 9.6 |

### Example 4

1. A liquid foundation using starch as a filler, including the following composition ingredients:

| | |
|---|---|
| Stearic acid | 3 parts |
| White mineral oil | 10 parts |
| Glycerol monostearate | 3 parts |
| Lanolin | 2 parts |
| P-hydroxyacetophenone | 0.2 part |
| Triethanolamine | 2 parts |
| Propylene glycol | 2 parts |
| Sodium carboxymethyl cellulose | 0.1 part |
| Deionized water | 60.7 parts |
| Titanium dioxide | 7 parts |
| Inorganic colorant | 1 part |
| Starch filler | 9 parts |

2. A preparation method of example 4 includes the following steps:

1) The raw materials were weighed according to the formula, and the water phase and oil phase raw materials were heated to 70° C. to completely dissolve the raw materials;

2) The oil phase was insulated at 70° C., and a toner powder mixed evenly was added under stirring;

3) The water phase raw materials were added under high-speed stirring and mixed evenly for 15 min, and the mixture was cooled to room temperature; and

4) Starch was added at 20° C., and the mixture was stirred for 20 min, and discharged and packed after stirring.

3. The relevant properties of Example 4 were measured. As shown by data in Table 12 and FIG. 1, the apparent viscosity is 1670 mPa·s, and shown by static rheological fitting data, the liquid foundation obtained in Example 4 is shear-thinned, has a fluid index of 0.1775 and a relatively large consistency coefficient K within the limit, and has slightly poor fluidity, shear resistance and strong adhesion.

TABLE 12
Properties of emulsified liquid foundation sample

| | Rheological properties | | | |
|---|---|---|---|---|
| | Apparent | Consistency | | Coefficient of |
| | viscosity/ | coefficient K/ | Rheological | determination |
| Sample | mPa · s | (Pa · s) | index n | R² |
| Example 4 | 1670 | 31.214 | 0.1775 | 0.9464 |

4. A preparation method of the starch filler in Example 4 includes: under the reaction conditions of pH 8.4 and 35° C., 8 wt % of an OSA reagent was added to 35% (w/w, d.b) starch milk to react for 4 h; and the mixture was neutralized, washed, micronized using a high-pressure homogenizer, homogenized 40 times under a pressure of 40 MPa, centrifugally dried, pulverized and sieved to obtain compound modified starch for the filler. The filler in Example 4, that is, the compound modified starch, was characterized for the degree of modification with the results shown in Table 3, and measured for the relevant properties with the results shown in Table 13. It can be seen that the degree of substitution of OSA is 0.03815 and the average particle size is 7.94 μm.

TABLE 13
Relevant properties of compound modified starch for filler

| | Gelatinization characteristic | | | |
|---|---|---|---|---|
| | Gelatinization | Peak viscosity/ | Final viscosity/ | Contact |
| Sample | temperature/° C. | (mPa · s) | (mPa · s) | angle/° |
| Example 4 | 57.3 | 4010 | 1452 | 64.00 |

5. To further illustrate the beneficial effects of the disclosure, the liquid foundation sample was subjected to sensory evaluation, and the evaluation scores were shown in Table 14. It can be seen that the liquid foundation using starch as a filler prepared by the disclosure is close to the control sample in performance, and can basically reach the standard of the commercial product.

TABLE 14
Sensory evaluation scores of emulsified liquid foundation samples

| | | Skin | Concealing | Trans- | Bright- |
|---|---|---|---|---|---|
| | Applicability | adhesion | effect | parency | ness |
| Example 4 | 11.1 | 10.5 | 10.8 | 9.0 | 10.4 |
| Control | 10.8 | 10.0 | 9.5 | 8.9 | 9.6 |

### Comparative Example 1

A preparation method of a starch filler includes: under the reaction conditions of pH 8.5 and 35° C., 5 wt % of an OSA reagent was added to starch milk to react for 4 h; and the mixture was neutralized, washed, dried, pulverized and sieved to obtain OSA modified starch. By measurement, the degree of substitution of OSA is 0.0263, and the average particle size is 15.93 μm.

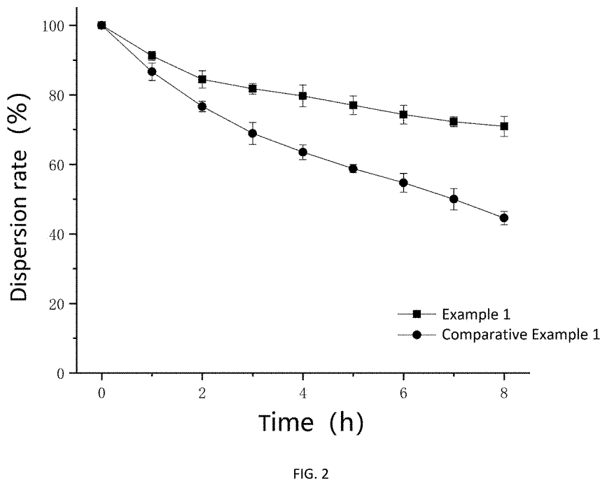

The composition of the liquid foundation is the same as that of Example 1. The OSA modified starch prepared in this comparative example was used as the filler, and the liquid foundation was prepared by the method of Example 1. The relevant properties of Comparative Example 1 were measured. The apparent viscosity and the rheological properties are shown by data in Table 15, the apparent viscosity is 1311 mPa·s, and shown by static rheological fitting data, Comparative Example 1 has little difference from Example 1 in static rheological properties. However, there is a certain gap between the sensory evaluation results of Comparative Example 1 and Example 1, and the evaluation scores are shown in Table 16. By comparison, the scores of the liquid foundation sample of Comparative Example 1 are generally lower than those of Example 1, especially in terms of applicability, skin adhesion and transparency, and the sample has a coarse texture and poor applicability. Moreover, as shown in FIG. 2, the dispersibility of the starch filler of Comparative Example 1 in the oil phase is lower than that of Example 1, indicating that Example 1 has better stability in an oil phase dispersion system as well as better storage stability.

TABLE 15
Properties of emulsified liquid foundation samples

| | | Rheological properties | | |
|---|---|---|---|---|
| | Apparent | Consistency | | Coefficient of |
| | viscosity/ | coefficient | Rheological | determination |
| Sample | mPa · s | K/(Pa · s) | index n | R² |
| Example 1 | 1255 | 12.724 | 0.3282 | 0.9801 |
| Comparative | 1311 | 11.948 | 0.2815 | 0.9510 |
| Example 1 | | | | |

TABLE 16
Sensory evaluation scores of emulsified liquid foundation samples

| | | Skin | Concealing | Trans- | Bright- |
|---|---|---|---|---|---|
| | Applicability | adhesion | effect | parency | ness |
| Example 1 | 10.5 | 9.4 | 8.9 | 9.3 | 11.2 |
| Comparative | 8.5 | 8.5 | 9.1 | 7.5 | 10.5 |
| Example 1 | | | | | |

### Comparative Example 2

The composition of the liquid foundation is same as Example 1.

A preparation method includes:

1) The raw materials were weighed according to the formula, and the water phase and oil phase raw materials were heated to 70° C. to completely dissolve the raw materials;

2) The oil phase was insulated at 75° C., and a toner powder mixed evenly and the starch filler were added under stirring; and

3) The water phase raw materials were added under high-speed stirring slowly and mixed evenly for 10 min, and the mixture was cooled to room temperature, discharged and packed.

The relevant properties of Comparative Example 2 were measured. As shown by data in Table 17, the apparent viscosity is 1075 mPa·s, which is slightly smaller than that of Example 1. Also, shown by static rheological fitting data, the sample of Comparative Example 2 is shear-thinned, has a consistency coefficient K also smaller than that of Example 1, and a fluid index with little difference.

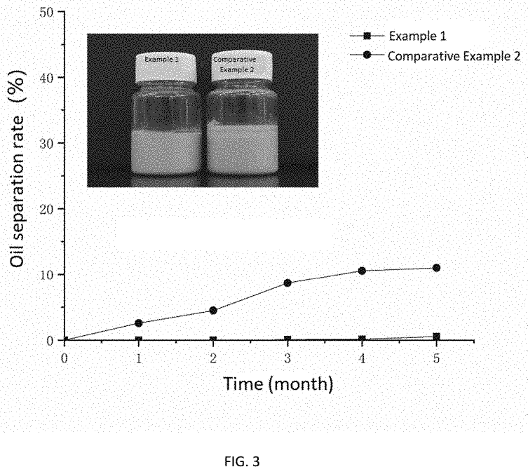

The sensory evaluation scores of Comparative Example 2 are shown in Table 18. By comparison, the sample of Comparative Example 2 has lower scores in applicability, concealing effect and brightness, mainly because in the preparation process of Comparative Example 2, the starch was gelatinized by heat, had no complete particle shape, and could not spread or adhere to the skin surface or play a role of light scattering. Moreover, FIG. 3 shows that, with the increase of time, the oil separation rate of the sample of Comparative Example 2 was much faster than that of Example 2, indicating that the storage stability of Comparative Example 2 was lower than that of Example 1.

TABLE 17
Properties of emulsified liquid foundation samples

| | | Rheological properties | | |
|---|---|---|---|---|
| | Apparent | Consistency | | Coefficient of |
| | viscosity/ | coefficient | Rheological | determination |
| Sample | mPa · s | K/(Pa · s) | index n | R² |
| Example 1 | 1255 | 12.724 | 0.3282 | 0.9801 |
| Comparative | 1075 | 10.887 | 0.2931 | 0.9510 |
| Example 2 | | | | |

TABLE 18
Sensory evaluation scores of emulsified liquid foundation samples

| | | Skin | Concealing | Trans- | Bright- |
|---|---|---|---|---|---|
| | Applicability | adhesion | effect | parency | ness |
| Example 1 | 10.5 | 9.4 | 8.9 | 9.3 | 11.2 |
| Comparative | 8.1 | 9.8 | 6.5 | 8.5 | 9.6 |
| Example 2 | | | | | |

Although the disclosure has been disclosed as above in the preferred examples, it is not intended to limit the disclosure. Any person skilled in the art can make various changes and modifications without departing from the spirit and scope of the disclosure. Therefore, the protection scope of the disclosure should be as defined in the claims.

What is claimed is:

1. A liquid foundation using starch as a filler, comprising the following ingredients in parts by mass:
10-20 parts oil phase, 65-75 parts water phase, 5-10 parts toner, 5-15 parts starch filler;
wherein the OSA degree of substitution of the starch filler is 0.02-0.05, and the average particle size is 5-12 μm.

2. The liquid foundation claim 1, wherein the composition of water phase raw materials comprises the following ingredients in parts by mass: 0.1-0.5 part of p-hydroxyacetophenone, 1-3 parts of triethanolamine, 2-4 parts of propylene glycol, 0.1-0.5 part of sodium carboxymethyl cellulose and 57-71.8 parts of water; the composition of the oil phase comprises the following ingredients in parts by mass: 1-3 parts of stearic acid, 1-3 parts of glycerol monostearate, 0.5-2 parts of lanolin, and 2-17.5 parts of white mineral oil; and the composition of the toner comprises the following ingredients in parts by mass: 0.5-2 parts of an inorganic colorant and 3-9.5 parts of titanium dioxide.

3. The liquid foundation of claim 1, wherein the starch filler is obtained by modifying starch with an OSA reagent and then performing homogenization and micronization, and the method specifically comprises: under the reaction conditions of pH 8.0-8.8 and 30-40° C., adding an OSA reagent which is 5-9% of the mass of starch to starch milk to react for 3-5 h; and after neutralizing and washing, micronizing the mixture using a high-pressure homogenizer, homogenizing the mixture 20-40 times under a pressure of 40-70 MPa, and centrifugally drying, pulverizing and sieving the mixture.

4. The liquid foundation of claim 2, wherein the inorganic colorant comprises one or more of iron oxide yellow, iron oxide red, and iron oxide black.

5. The liquid foundation of claim 4, wherein when the inorganic colorant comprises iron oxide yellow and iron oxide red, the mass ratio of the iron oxide yellow to the iron oxide red is 4:1.

6. The liquid foundation of claim 3, wherein the starch is any one or more of corn starch, wheat starch, potato starch, tapioca starch and corresponding waxy starch thereof.

7. The liquid foundation of claim 6, wherein the concentration of the starch milk is 30-40% (w/w, d.b).

8. A preparation method of the liquid foundation of claim 1, comprising the following steps:
1) weighing raw materials according to the formula, and heating the water phase and oil phase raw materials to 75-80° C. to completely dissolve the raw materials;
2) under a stirring state at 70-75° C., adding a toner powder mixed evenly to the oil phase;
3) then adding the water phase raw materials under high-speed stirring, mixing the raw materials evenly for 10-20 min, and cooling the mixture to 20-30° C.; and
4) adding a starch filler at 20-30° C., stirring the mixture for 5-10 min, and then performing discharging and packing after stirring.
